(19) United States
(12) Patent Application Publication
(10) Pub. No.: US 2001/0031052 A1
(43) Pub. Date: Oct. 18, 2001

(54) Noise suppression loudspeaker

(76) Inventors: Christopher Colin Lock, Auckland (NZ); John Frederick Corey, Auckland (NZ); Graeme Oliver Jones, Auckland (NZ); Kelly Charles Waterman, Waitakere City (NZ); Mark Donaldson, Alfriston (NZ)

Correspondence Address: YOUNG & THOMPSON, 745 SOUTH 23RD STREET 2ND FLOOR, ARLINGTON, VA, 22202

(21) Appl. No.: 09/799,733

(22) Filed: Mar. 7, 2001

(30) Foreign Application Priority Data
Mar. 7, 2000 (NZ) 503222

(51) Int. Cl. A61F 11/06; G10K 11/16; H03B 29/00; H04R 25/00

(52) U.S. Cl. 381/71.7; 381/71.1; 381/71.6; 381/152

(57) ABSTRACT

A loudspeaker system is disclosed for active noise reduction. The loudspeaker has a substantially flat or planar diaphragm which is vibrated by an appropriate driver to generate a cancelling signal to cancel noise in the vicinity of the loudspeaker. The loudspeaker includes an input transducer such as a microphone which detects the ambient noise. The microphone is preferably incorporated in the panel structure of the loudspeaker in such a way that the ambient noise information can be captured and presented to active noise cancelling circuitry. The circuitry then provides an appropriate signal to the loudspeaker driver so that the cancelling sound source is provided by the loudspeaker. The use of flat panel loudspeaker technology has been found to provide effective noise reduction and is very appropriate for circumstances in which noise reduction is desirable, for example in aircraft, bus or vehicle seats or telephone kiosks.

### BACKGROUND

Loudspeakers with flat or planar diaphragms have been known for a number of years. There have been many attempts to create flat panel speakers in order to save space such as the well known electrostatic speakers which can be hug from a wall like a picture. Many situations where noise suppression is used or is most desirable involve relatively confined spaces such as the aircraft cabs, motor vehicles and other environments where space is a premium. The use of flat panel speakers in such situations would be a decided advantage over conventional cone type speakers.

### OBJECT OF THE INVENTION

It is an object of the present invention to provide a loudspeaker for use with noise suppression systems which will at least go some way toward overcoming the disadvantages or limitations of known speakers, or to at least provide the public with a useful choice.

### SUMMARY OF THE INVENTION

In one aspect, the invention resides in a loudspeaker including in combination a planar diaphragm which can be vibrated to radiate sound, a driver unit attached to the diaphragm in one or more selected positions, the driver unit caused by varying electric current to vibrate the diaphragm, the electric current varied by an active noise suppression apparatus whereby in operation noise cancellation can be achieved when operating the loudspeaker.

Preferably the diaphragm is formed from a sheet of material.

Preferably the sheet material has a bending strength in a single plane along a first axis which is greater than along a second axis which is perpendicular to the first axis.

Preferably the sheet material is flexible but has stiffening means to prevent the diaphragm from collapsing from its own weight.

Preferably the stiffening means comprises a cellular structure of the sheet material. In the alternative where sheet material without a cellular structure is used, the stiffening means can include ridges, swages, or other stiffening sections moulded or attached to the diaphragm.

Preferably the sheet material is core flute or corrugated cardboard, however, other material such as injection moulded or extruded plastic having a flat configuration may be used.

Preferably the core flute or corrugated cardboard has a cellular structure comprising longitudinal cells which have a square or substantially square cross section.

Preferably the diaphragm when formed from core flute or corrugated cardboard is between 3 and 8 millimeters thick and most preferably about 5 millimeters thick.

In an alternative version, the diaphragm can be constructed of a resilient plastic such as high density polypropylene or equivalent material of uniform consistency and thickness.

In the alternative the diaphragm may be of non-uniform consistency or thickness in selected regions to improve or tune the loudspeaker's frequency response. The diaphragm may also have moulded regions of ribs, grooves or other patterns to improve or tune the loudspeaker's frequency response.

Suitably the diaphragm is rectangular, square or rounded square or oval shaped or any combination thereof.

Preferably there is a single driver unit however two or more driver units may be placed at selected positions on the diaphragm to achieve a desired vibrational response of the diaphragm.

Preferably the driver unit comprises a coil assembly which is non-linear in its electrical response.

Preferably the weight of the driver unit is at least between 50 to 100 grams and ideally around 60 grams.

Preferably the magnet of the driver unit is a neodymium iron/boron sinter magnet selected for its power and mass. Preferably the weight of the magnet is about 20 grams however heavier magnets can be used.

Preferably the driver unit is mounted by a damper sealer to the diaphragm.

Preferably the damper sealer neoprene or rubber.

Preferably the driver unit is located off centre with respect to the diaphragm in order to improve the frequency response of the loudspeaker.

In the alternative the driver unit can be mounted in a separate enclosure which is then attached to the diaphragm in a quasi bi-planar configuration wherein the enclosures responds to internal frequencies and transmits sounds to the rear of the diaphragm.

Preferably the quasi bi-planar enclosure should be distanced sufficiently away from the diaphragm to create a non-ported sealed chamber.

Preferably a frame or peripheral chassis surrounds the diaphragm. Alternatively the frame or chassis can comprise two or more frame members attached to the sides or edges of the diaphragm.

Where the frame or chassis surrounds the diaphragm, there can also be attached to the frame or chassis a quasi bi-planar rear panel which together with the diaphragm forms an enclosure for the driver unit.

Preferably the rear panel is formed of sheet material which responds to the internal frequencies of the enclosure so formed and vibrates to transmit sound away from the diaphragm. The panel may or may not have apertures to improve the acoustic performance of the loudspeaker.

Preferably the frame or chassis is insulated from the diaphragm by means of a damper of foam or other suitable material.

In the alternative, the damper can comprise a combination of hard and soft materials to variably insulate the diaphragm from the frame or chassis.

In another version of the invention, the driver unit can be suspended from the frame or chassis by means of a movable yoke attached to a frame, the yoke being substantially parallel to the diaphragm to allow the driver unit to transfer its momentum and inertial energy to the diaphragm without the, diaphragm actually supporting the weight of the driver unit.

Preferably the yoke is stainless steel wire, however other yokes of resilient plastic or equivalent materials can be used.

In another aspect, the invention resides in a loudspeaker substantially as herein before described with the inclusion of cordless means adapted to enable the loudspeaker to be operated without leads or wires from a current source.

Preferably the cordless means comprises a radio frequency signal receiver with complementary power amplification means to provide varying electric current to the driver unit corresponding to the signals received.

Suitably there is a complementary radio sign generation and transmission means to generate and transmit the signals received by the signal receiver. Suitably the signal generation and transmission means is coupled to a audio amplifier connected to a source of sound or music.

Preferably the noise suppression apparatus includes an open air transducer panel, microphone and noise cancelling circuitry.

The loudspeaker and noise suppression apparatus can be installed in applications such as a head rest or kiosk structure capable of partially enveloping the head of the listener in the sound field of one or more loudspeaker. In the alternative the unit may be built into the headrest wings of an aircraft seat, or into the headrest and backrest of other seating systems used in mass transport.

In another version the apparatus can be designed to fit the structure of a telephone kiosk or other information kiosk where in the head of the listener is partially surrounded by loudspeakers and in the sound field produced by the loudspeakers.

The environment in which this apparatus operates may be harsh and require the apparatus to be robust and withstand exposure to:

Ultraviolet light,

ozone and

other environmental pollutants as well as

wide variations in a temperature and humidity.

The invention described incorporates above, mentioned flat loudspeaker technology, which is used to generate the cancelling signal for the ambient noise. An input transducer or microphone which detects the ambient noise can be incorporated in the panel structure or be incorporated in the apparatus structure in such a way that the ambient noise information can be capture and presented to the active noise cancelling circuitry. Use of flat panel technology is an ideal mechanism to produce the dispersive sound pressure waves, which are used to cancel the ambient noise in the near field.

The noise cancelling circuitry takes the signal from the microphone and produces an appropriate output signal to drive, the panel transducer, producing the necessary cancelling waves. This circuitry can utilise either nonfeedback open loop or closed loop feedback techniques to produce a cancelling signal, and can be implemented in either digital or analogue circuits. Preferably the noise cancelling circuitry will be located in an adjacent electronics enclosure with a power supply.

Additionally other signals can be presented to the apparatus to be summed with the noise cancelling signal, such as music~or public address sound sources. These signals can also be provided to the noise cancelling circuitry as a reference which is masked from the ambient noise input so that they will not be suppressed by the noise cancelling function.

### BRIEF DESCRIPTION OF THE DRAWINGS

In order to better understand the, invention reference will now be made to the, accompanying drawings wherein:

FIGS. 1a and 1b: are perspective and cross sectional views of the invention according to Example 1.

FIGS. 2a and 2b: show various applications of the invention of Example 1.

FIG. 3: is an example of a schematic diagram of an active noise reduction system that may be used according to the present invention.

### DETAILED DESCRIPTION OF THE INVENTION

### Example 1

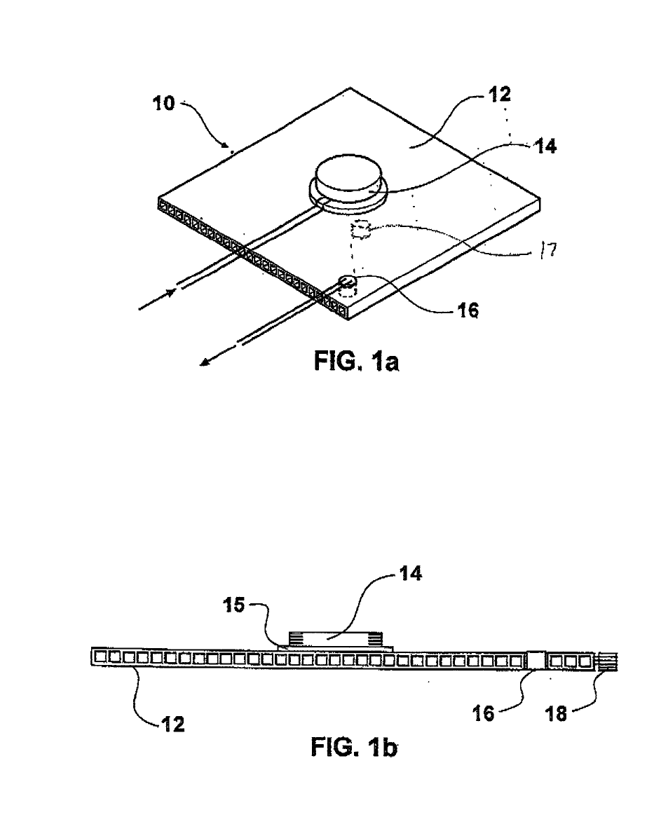

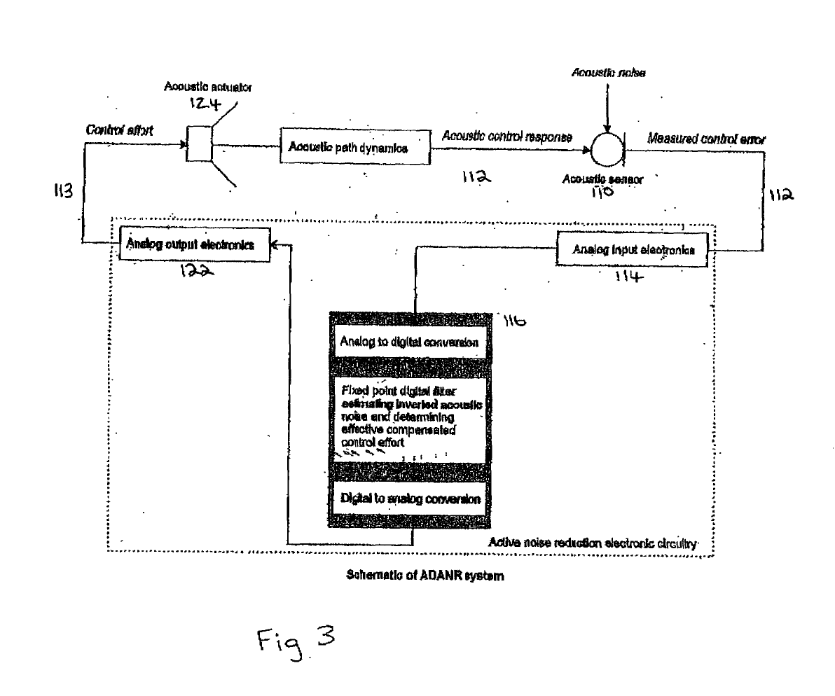

Referring now to FIG. 1a there is shown a flat or planar loudspeaker generally referenced 10 incorporating noise cancellation apparatus according to Example 1. There is shown a planar loudspeaker 10 having a diaphragm 12 on which there is located a microphone 16 which detects ambient noise. Ambient noise detected by microphone 16 is provided to the noise cancelling circuitry (not shown) as discussed further below with reference to FIG. 3. The noise cancelling circuitry then produces a cancelling signal which is then sent to the transducer 14 of the speaker which causes the speaker panel or diaphragm to vibrate producing sound which causes destructive interference with the noise detected by the microphone 16. This results in cancellation of the ambient noise, so a zone of silence in the vicinity of the loudspeaker is produced. Typically, the microphone 16 will be located near the centre of the diaphragm in a position such as position 17, shown in FIG. 1a in dashed outline.

Referring now to FIG. 1b the planar loudspeaker incorporating the noise cancellation apparatus according to Example 1 is shown in cross section. FIG. 1b shows the core flute type panel material in more detail. The general construction of a loudspeaker having a substantially planar diaphragm, but not including a sensing means such as microphone 16 or any active noise reduction circuitry or system is disclosed in International Patent Application No. WO 99/67974, the disclosure of which is incorporated herein by reference.

The core flute material may be constructed from a variety of different constituent materials, for example plastics or fibrous materials such as corrugated cardboard. He material 12 may be formed in plastics as an integral extrusion of polyethylene around 3-5 mm thick and having upper and lower skis 13 and 17 joined by continuous longitudinal walls or webs 19 with air spaces therebetween. By this construction the material is substantially an isotropic and has a longitudinal bending strength which may typically be around twice the transverse bending strength. A similar construction and result may be realised using corrugated cardboard.

The microphone 16 is preferably an omnidirectional microphone which is sensitive to sound pressure waves in the audio spectrum. The omnidirectional microphone 16 has been found to provide good performance for picking up ambient noise. The omnidirectional microphone 16 is preferably placed at, in or on the surface of the panel so as to pick up sound energy including noise in the vicinity of the panel, and may be recessed into the panel as shown.

In the alternative the omnidirectional microphone which is sensitive to ambient noise can be located externally to the panel as shown by 18. The microphone may be placed in a variety of locations in the vicinity of the loudspeaker, for example in the middle of the planar diaphragm or adjacent to an edge thereof.

It is desirable that the omnidirectional microphone which is sensitive to the ambient noise to be cancelled is located near and in the plane of the loudspeaker so that the ambient noise is captured faithfully. In this way the microphone also captures sound and noise in the immediate vicinity of the loudspeaker to thereby ensure tat a region in the acoustic path or sound field near the loudspeaker, and thus probably near the ears of a listener, will be subject to effective noise suppression.

The transducer 14 is located with a damper 15 on the surface of the diaphragm 12. The damper is desirably comprised of a resilient material such as neoprene or rubber. Also, the transducer 14 may be mounted off-centre with respect to the diaphragm in order to improve the frequency response of the loudspeaker. The transducer 14 can also be mounted in a separate enclosure which is then attached to the diaphragm.

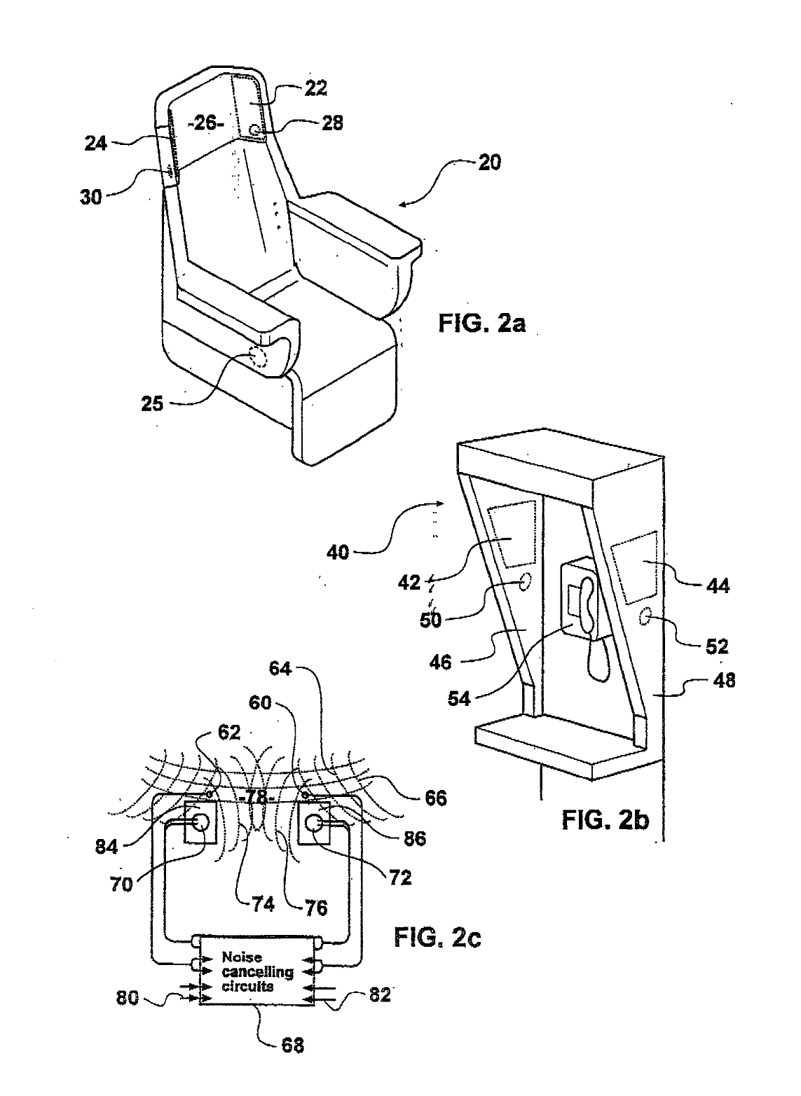

FIG. 2a is an isometric perspective of an airline or coach seat 20 showing how the invention is used. Planar speakers 22, 24 as hereinbefore mentioned are located in the cushions of the headrest 20 of the seat The planar loudspeakers include microphones 28, 30 which detect ambient noise which is fed by the noise cancelling circuitry to a noise cancellation unit 25 located in the seat. Ambient noise picked up in the region of the head of a passenger (not shown) is fed back to the noise cancellation circuitry wherein the noise cancelling signal is then radiated from the panel speakers thereby canceling the noise.

FIG. 2b shows the application of Example 1 in a telephone kiosk 40 wherein the loudspeaker panels 42, 44 are incorporated in the side enclosures 46, 48 of the booth.

Microphones 50, 52 detect ambient noise and feed his back by a noise cancellation circuit (not shown) to the panel loudspeakers 42, 44 thereby creating a noise cancelled environment around the telephone 54. Therefore, a user of the telephone, standing in the booth will experience a zone of relative silence or near silence when using the telephone as any background noise will be suppressed.

FIG. 2c is a schematic diagram showing the apparatus of the invention of Example 1 wherein microphones 60, 62 pick up ambient noise shown by broken lines 64, 66. The microphones are attached to the noise cancelling circuitry 68.

Noise cancelling signals are then sent to the transducers 70, 72 of the flat panel speakers whereby cancelling waves 74, 76 are produced to effect a noise cancelled region 78.

There may be auxiliary inputs 80, 82 of music or other sound which are used as reference values by he noise cancelling circuit and which are masked from the ambient noise so that it can be sent to the panel speakers 84, 86 without being effected by the noise cancellation system. In this way the loudspeakers of the present invention may be used to convey music, ad/or public address notices while also providing noise suppression.

In FIG. 3, a schematic diagram of a noise suppression system capable of effecting noise suppression using the loudspeaker of the present invention is shown.

In FIG. 3 an acoustic sensor 110 which may comprise microphone 16 of FIG. 1a, for example, is provided together with associated components such as cables and connectors 112 and appropriate analog input electronics 114.

The active noise reduction electronics shown in FIG. 3 incorporates the analogue input electronics 114, the digital-signal-processor and the analog-to-digital and digital-to-analog converters 116, and the analogue output electronics 122.

The flat panel loudspeaker is referenced 124 in FIG. 3, and has associated components such as cables and connectors 113).

The digital signal processor comprises a digital feedforward filter, preferably a fixed point filter, implemented physically on DSP, and determines an appropriate control effort based on the measured and sampled control error signal produced by the microphone 110.

The digital filter estimates inverted acoustic noise and from this determines the control effort which is compensated for undesirable dynamic components of the system. A feedforward control strategy is therefore provided which does not suffer from closed loop stability problems or poor parameter convergence associated with adaptive filter control systems.

### ADVANTAGES

It will be evident that a major advantage of the present invention is the ability to use existing noise suppression technology with a planar loudspeaker which does not occupy a lot of space. It is also an advantage that the loudspeaker panels are made from materials which are not affected by moisture, temperature, UV light, sunlight or other adverse environmental factors.

In addition planar loudspeakers are also lighter than their cone counterparts and this is particularly important where weight is an important consideration such as in aircraft.

### VARIATIONS

Finally, it will be appreciated that various other alterations and modifications may be made to the foregoing without departing from the scope of this invention as set forth.

Throughout the description and claims of this specification the word “comprise” and variations of that word, such as “comprises” and “comprising”, are not intended to exclude other additives, components, integers or steps.

1. A loudspeaker including in combination a planar diaphragm which can be vibrated to radiate sound, a driver unit attached to the diaphragm in one or more selected positions, the driver unit caused by varying electric current to vibrate the diaphragm, the electric current varied by an active noise suppression apparatus whereby in operation noise cancellation can be achieved when operating the loudspeaker.

2. A loudspeaker as claimed in claim 1 wherein the diaphragm is formed from a sheet of material.

3. A loudspeaker as claimed in claim 2 wherein the sheet material has a bending strength in a single plane along a first axis which is greater than along a second axis which is perpendicular to the first axis.

4. A loudspeaker as claimed in claim 2 wherein the sheet material is flexible but has stiffening means to prevent the diaphragm from collapsing from its own weight.

5. A loudspeaker as claimed in claim 4 wherein the stiffening means comprises a cellular structure of the sheet material. In the alternative where sheet material without a cellular structure is used, the stiffening means can include ridges, swages or other stiffening sections moulded or attached to the diaphragm.

6. A loudspeaker as claimed in claim 4 wherein the sheet material is core flute or corrugated cardboard, however, other material such as injection moulded or extruded plastic having a flat configuration may be used.

7. A loudspeaker as claimed in claim 6 wherein the core flute or corrugated cardboard has a cellular structure comprising longitudinal cells which have a square or substantially square cross section.

8. A loudspeaker as claimed in claim 1 wherein the driver unit is mounted by a damper sealer to the diaphragm.

9. A loudspeaker as claimed in claim 1 wherein the driver unit is located off-center with respect to the diaphragm in order to improve the frequency response of the loudspeaker.

10. A loudspeaker as claimed in claim 1 wherein the driver unit is mounted in a separate enclosure which is then attached to the diaphragm in a quasi bi-planar configuration wherein the enclosures responds to internal frequencies and transmits sounds to the rear of the diaphragm.
